(19) United States
(12) Patent Application Publication
(10) Pub. No.: US 2026/0345722 A1
(43) Pub. Date: Nov. 5, 2026

(54) OCTREOTIDE ACETATE COMPOSITION AND METHODS OF MAKING DELAYED RELEASE ORAL DOSAGE FORMS

(71) Applicant: SpecGx LLC, Webster Groves, MO (US)

(72) Inventors: Jae Han Park, St. Louis, MO (US); Giselle Mujica, St. Louis, MO (US); Quinn Pazderka, St. Louis, MO (US); Gary Nichols, St. Louis, MO (US); Jeffery Carter, St. Louis, MO (US)

(21) Appl. No.: 19/188,823

(22) Filed: Apr. 24, 2025

(51) Int. Cl. A61K 38/08; A61K 9/48; A61K 9/50

(52) U.S. Cl. CPC A61K 38/08; A61K 9/485; A61K 9/4858; A61K 9/4866; A61K 9/4891; A61K 9/5042

(57) ABSTRACT

The present disclosure is directed to a composition, method of making the composition, and method of using the composition. The composition comprises a plurality of microcrystalline cellulose (MCC) spheres, an octreotide or a pharmaceutically acceptable salt of octreotide, a permeability enhancer, and a stabilizing agent. The composition further comprises an amino acid, a divalent salt, and a binder. The composition may be formulated as a capsule with an enteric coating.

### BACKGROUND

Acromegaly is a hormonal disorder that results when the pituitary gland produces excess growth hormone (GH). It most commonly affects middle-aged adults and can result in serious illness and premature death. Once recognized, acromegaly is treatable in most patients, but because of its slow and often insidious onset, it frequently is not diagnosed correctly. Octreotide is one drug used to treat acromegaly. Octreotide exerts pharmacologic actions similar to those of the natural hormone somatostatin. Octreotide decreases GH and IGF-1 levels, as well as glucagons and insulin. Octreotide also suppresses luteinizing hormone (LH) response to gonadotropin releasing hormone (GnRH), decreases splanchnic blood flow, and inhibits the release of serotonin, gastrin, vasoactive intestinal peptide, secretin, motilin, and pancreatic polypeptide. Octreotide may be administered as an injectable or an oral dosage form.

The only currently available oral dosage form of octreotide is a delayed release capsule. The delayed release capsule is formulated as an oily suspension of lyophilized octreotide. The current commercial product requires non-conventional manufacturing processes including lyophilization of octreotide with excipients, manufacturing of an oily dispersion of lyophilized octreotide, encapsulation of the oily dispersion into hard gelatin capsules, and enteric polymer coating over the capsules.

Therefore, there exists a need for a non-oil dispersion-based composition of octreotide in a delayed release capsule that is bioavailable and demonstrates an acceptable stability.

### SEQUENCE LISTING

The present application contains a Sequence Listing which has been submitted electronically in .XML format and is hereby incorporated herein by reference in its entirety. Said computer readable file, was created on Apr. 24, 2025 is named 054501-838508-SequenceListing. XML and is 296 kilobytes in size.

### BRIEF SUMMARY

The present disclosure is directed to a delayed release composition. The delayed release composition comprises a plurality of spheres; an octreotide or a pharmaceutically acceptable salt of octreotide; a permeability enhancer; a stabilizing agent; a divalent salt; and a binder.

The present disclosure is also directed to a composition. The composition comprises a peptide or a pharmaceutically acceptable salt of a peptide; and an amino acid comprising leucine, cysteine, aspartate, phenylalanine, glutamine, or a combination thereof, wherein the ratio of the peptide or salt thereof to amino acid is 1:1 to 50:1, 2:1 to 50:1, 3:1 to 50:1, or 4:1 to 50:1 on a w/w basis.

The present disclosure is directed to a method of making a delayed release composition. The method comprises preparing a plurality of spheres; preparing a first coating solution comprising an amino acid, a divalent salt, a binder, octreotide or a pharmaceutically acceptable salt of octreotide, and a permeability enhancer; coating the plurality of spheres with the first coating solution in a fluid bed unit; preparing a second coating solution comprising a stabilizing agent, the binder, and a nonionic surfactant, and divalent salt; coating the plurality of spheres coated with the first coating solution with the second coating solution; encapsulating the coated plurality of spheres into a capsule; preparing an enteric coating solution; and coating the capsule with the enteric coating solution.

### DETAILED DESCRIPTION

The present disclosure is directed to a delayed release octreotide composition and methods of making thereof. The octreotide composition may be a non-oil dispersion-based composition of octreotide in a delayed release solid dosage composition. In some embodiments the composition may be formulated as a delayed release solid oral dosage form. Delayed release solid dosage forms may include dosage forms such as tablets, capsules and particulate formulations. In some embodiments the delayed release dosage forms may be enterically coated. The delayed release composition may exhibit reduced impurities as compared to other octreotide compositions. Impurities may include degradation products. The composition may be a pharmaceutical composition. In various embodiments, the composition may be used for long-term treatment of acromegaly in patients. In some embodiments, the patients treated with the composition may have previously responded to octreotide and lanreotide injections.

Before the present compositions and methods are described, it is to be understood that this disclosure is not limited to the particular molecules, compositions, methodologies or protocols described, as these may vary. It is also to be understood that the terminology used in the description is for the purpose of describing the particular versions or embodiments only and is not intended to limit the scope of the present disclosure which will be limited only by the appended claims. The terms used herein have meanings recognized and known to those of skill in the art, however, for convenience and completeness, particular terms and their meanings are set forth below.

It must also be noted that as used herein and in the appended claims, the singular forms “a”, “an”, and “the” include plural reference unless the context clearly dictates otherwise. Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the preferred methods, devices, and materials are now described. All publications mentioned herein are incorporated by reference to the extent they support the present disclosure. Nothing herein is to be construed as an admission that the disclosure is not entitled to antedate such disclosure by virtue of prior disclosure.

The term “pharmaceutically acceptable salts” refers to the relatively non-toxic, inorganic and organic acid addition salts of compounds of the present disclosure. These salts may be prepared in situ during the final isolation and purification of the compounds or by separately reacting the purified compound in its free base form with a suitable organic or inorganic acid and isolating the salt thus formed. Representative salts may include the acetate, hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. These may include cations based on the alkali and alkaline earth metals, such as sodium, lithium, potassium, calcium, magnesium, and the like, as well as non-toxic ammonium, tetramethylammonium, tetraethylammonium, methlyamine, dimethlyamine, trimethlyamine, triethlyamine, ethylamine, and the like.

As used herein, the term “about” means plus or minus 10% of the numerical value of the number with which it is being used. For example, about 50% means in the range of 45%-55%.

As used herein, the term “delayed release” refers to a type of drug delivery system designed to release the active ingredient of a medication at a specific time or in a particular part of the digestive system, rather than immediately upon ingestion. Delayed release compositions or formulations may be used to avoid irritation of the stomach lining, protect the drug from stomach acid, or ensure that the drug is released where it is most effective (e.g., in various regions of the intestine). One example of a delayed release formulation may include enteric-coated capsules. The enteric coating prevents the capsule from dissolving in the acidic environment of the stomach but allows it to dissolve in the more neutral or alkaline environment of the intestine.

As used herein, the term “enteric coating” refers to coatings that prevent peptide degradation in the acidic stomach and release the peptide only in the more neutral pH environment of the small intestine.

### Composition

Octreotide acetate is a somatostatin analogue indicated for long-term maintenance treatment in acromegaly patients who have responded to and tolerated treatment with octreotide or lanreotide.

Parenteral compositions (injection and infusion) of octreotide acetate are used: to manage severe diarrhea and flushing in symptomatic patients with metastatic carcinoid tumors; to treat profuse watery diarrhea associated with vasoactive intestinal peptide (VIP) tumors; to reduce growth hormone (GH) and insulin-like growth factor 1 [(IGF-1), known as somatomedin C] in patients with acromegaly who have not responded adequately to, or cannot be treated with surgical resection, pituitary irradiation or bromocriptine mesylate at maximally tolerated doses; as an adjuvant treatment with pituitary irradiation to reduce GH and IGF-1 level in patients with acromegaly; a symptomatic treatment of acromegaly patients; to reduce the incidence and severity of post-operative complications following pancreatic surgery; and in combination with sclerotherapy (or other interventions) to control gastro-esophageal varices bleeding due to underlying cirrhosis.

Octreotide has similar pharmacological effects to naturally occurring somatostatin, but more potently inhibits growth hormone (GH), glucagon, and insulin. Octreotide, like somatostatin, also suppresses the response of luteinizing hormone (LH) to gonadotrophin-release hormone (GnRH), decreases splanchnic blood flow, and inhibits the release of serotonin, gastrin, vasoactive intestinal peptide, secretin, motilin, and pancreatic polypeptide. In healthy subjects, octreotide acetate has been shown to inhibit release of GH stimulated by arginine infusion, exercise, and insulin-induced hypoglycemia; postprandial release of insulin, glucagon, gastrin, other peptides of the gastro-entero-pancreatic (GEP) endocrine system, and arginine-stimulated release of insulin and glucagon; and/or Thyrotropin releasing hormone (TRH) stimulated release of TSH. The exact mode of action of octreotide acetate on portal hypertension is still unclear. The inhibition of vasoactive gastro-intestinal hormone secretion and exertion of a direct vasomotor effect on splanchnic vessels, consequently reducing portal blood flow is thought to mainly cause the reduction of the splanchnic blood flow. It has been shown using human saphenous veins, that vasoconstriction, is mediated by type 2 somatostatin receptors.

In many patients, GH levels fall within one hour and headaches improve within minutes after the injection of octreotide. Several studies have shown that octreotide may be effective for long-term treatment of acromegaly. Octreotide also has been used successfully to treat patients with acromegaly caused by non-pituitary tumors. In some acromegaly patients who already have diabetes, octreotide can reduce the need for insulin and improve blood sugar control.

Peptides, being relatively small chains of amino acids, may be sensitive to environmental factors such as temperature, pH, and oxidation. These factors can cause degradation, resulting in a loss of therapeutic activity. Peptides may be broken down in the gastrointestinal tract by enzymes before they enter the bloodstream, resulting in their poor absorption. Because of their poor absorption, peptides are typically administered via injection instead of orally.

Examples of orally administered peptides include but are not limited to oral composition of octreotide sold under the trademark Mycapssa®, semaglutide tablet sold under the trademark Rybelsus®, and oral insulin formulations.

The only currently approved delayed release oral composition of octreotide is Mycapssa®. The approved delayed release oral composition of octreotide under the trademark Mycapssa® is formulated as an oily dispersion using transient permeation enhancer (TPE) technology combined with an enteric coating. Oily dispersions may provide one way to orally administer peptides. There exists a need for a non-oil dispersion-based composition of octreotide in a delayed release capsule that is bioavailable and demonstrates an acceptable stability.

Provided herein are oral delayed release compositions of octreotide with improved stability and bioavailability. In various embodiments, the compositions are not oil dispersions.

In some embodiments, the composition may include a plurality of particles. In some embodiments, the particles may comprise spheres or granules. In additional embodiments, the particles may include microcrystalline cellulose (MCC) spheres, sugar spheres, or any other suitable particles. In further embodiments, the plurality of particles may comprise a plurality of any suitable delayed release beads.

In various embodiments, the composition may include microcrystalline cellulose (MCC) spheres, an octreotide or a pharmaceutically acceptable salt of octreotide, a permeability enhancer, and a stabilizing agent. The composition may further include an amino acid, a divalent salt, a binder, a nonionic surfactant, one or more peptides or any combination thereof. In various embodiments the composition may be formulated as a capsule. In some examples, the composition may have an enteric coating. In additional examples, the composition may be a delayed release capsule composition. In some examples, the composition may comprise delayed release particulates or beads.

In some embodiments, the size of the particles in the composition may range from 100 μm to 900 μm. In some embodiments, the size may be from about 100 μm to about 900 μm, about 100 μm to about 850 μm, about 100 μm to about 800 μm, about 100 μm to about 750 μm, about 100 μm to about 700 μm, about 100 μm to about 650 μm, about 100 μm to about 600 μm, about 100 μm to about 550 μm, about 100 μm to about 500 μm, about 100 μm to about 450 μm, about 100 μm to about 400 μm, about 100 μm to about 350 μm, about 100 μm to about 300 μm, about 100 μm to about 250 μm, about 100 μm to about 200 μm, or about 100 μm to about 150 μm. In some embodiments, the size may be from about 100 μm to about 200 μm.

In some embodiments, the size may be from 100 μm to 900 μm, 100 μm to 850 μm, 100 μm to 800 μm, 100 μm to 750 μm, 100 μm to 700 μm, 100 μm to 650 μm, 100 μm to 600 μm, 100 μm to 550 μm, 100 μm to 500 μm, 100 μm to 450 μm, 100 μm to 400 μm, 100 μm to 350 μm, 100 μm to 300 μm, 100 μm to 250 μm, 100 μm to 200 μm, or 100 μm to 150 μm. In some embodiments, the size may be from 100 μm to 200 μm.

In some embodiments, the composition may be in an enteric-coated oral dosage form. The enteric-coated dosage form may include a plurality of spheres, an octreotide or a pharmaceutically acceptable salt of octreotide, a permeability enhancer, and a stabilizing agent. In some embodiments, the spheres may be microcrystalline (MCC) spheres.

MCC spheres are an excipient that may be used as a filler in the pharmaceutical compositions. Alternatives to MCC spheres may include but are not limited to sugar spheres or any other suitable particles or beads.

MCC spheres may comprise small, spherical particles made from microcrystalline cellulose and water. In some examples, the MCC spheres may be produced by any suitable spherization process. In some embodiments comprising MCC spheres, the size of the microcrystalline cellulose (MCC) spheres may range from 100 μm to 900 μm. In some embodiments, the size may be from about 100 μm to about 900 μm, about 100 μm to about 850 μm, about 100 μm to about 800 μm, about 100 μm to about 750 μm, about 100 μm to about 700 μm, about 100 μm to about 650 μm, about 100 μm to about 600 μm, about 100 μm to about 550 μm, about 100 μm to about 500 μm, about 100 μm to about 450 μm, about 100 μm to about 400 μm, about 100 μm to about 350 μm, about 100 μm to about 300 μm, about 100 μm to about 250 μm, about 100 μm to about 200 μm, or about 100 μm to about 150 μm. In some embodiments, the size may be from about 100 μm to about 200 μm.

In some embodiments, the size may be from 100 μm to 900 μm, 100 μm to 850 μm, 100 μm to 800 μm, 100 μm to 750 μm, 100 μm to 700 μm, 100 μm to 650 μm, 100 μm to 600 μm, 100 μm to 550 μm, 100 μm to 500 μm, 100 μm to 450 μm, 100 μm to 400 μm, 100 μm to 350 μm, 100 μm to 300 μm, 100 μm to 250 μm, 100 μm to 200 μm, or 100 μm to 150 μm. In some embodiments, the size may be from 100 μm to 200 μm.

The weight percent of the particles in the composition may be from 10% w/w to 60% w/w of the total weight of the composition. The weight percent of the particles may be from 30% w/w to 50% w/w of the total weight of the composition. In various embodiments, the weight percent of the particles may be from about 10% w/w to about 60% w/w, from about 20% w/w to about 60% w/w, from about 20% w/w to about 50% w/w from about 30% w/w to about 60% w/w, from about 30% w/w to about 50% w/w, about 30% w/w to about 49% w/w, about 30% w/w to about 48% w/w, about 30% w/w to about 47% w/w, about 30% w/w to about 46% w/w, about 30% w/w to about 45% w/w, about 30% w/w to about 44% w/w, about 30% w/w to about 43% w/w, about 30% w/w to about 42% w/w, about 30% w/w to about 41% w/w, about 30% w/w to about 40% w/w, about 30% w/w to about 39% w/w, about 30% w/w to about 38% w/w, about 30% w/w to about 37% w/w, about 30% w/w to about 36% w/w, about 30% w/w to about 35% w/w, about 30% w/w to about 34% w/w, about 30% w/w to about 33% w/w, about 30% w/w to about 32% w/w, or about 30% w/w to about 31% w/w of the total weight of the composition. In some embodiments, the weight percent of the particles may be from about 30% w/w to about 40%, about 35% w/w to about 40%, about 38% w/w to about 42%, or about 39% w/w to about 40%. In some embodiments, the weight percent of the particles may be about 40% w/w of the total weight of the composition.

In some embodiments, the weight percent of the particles may be from 10% w/w to 60% w/w, from 20% w/w to 50% w/w from 20% w/w to 60% w/w, from 30% w/w to 60% w/w, from 30% w/w to 50% w/w, 30% w/w to 49% w/w, 30% w/w to 48% w/w, 30% w/w to 47% w/w, 30% w/w to 46% w/w, 30% w/w to 45% w/w, 30% w/w to 44% w/w, 30% w/w to 43% w/w, 30% w/w to 42% w/w, 30% w/w to 41% w/w, 30% w/w to 40% w/w, 30% w/w to 39% w/w, 30% w/w to 38% w/w, 30% w/w to 37% w/w, 30% w/w to 36% w/w, 30% w/w to 35% w/w, 30% w/w to 34% w/w, 30% w/w to 33% w/w, 30% w/w to 32% w/w, or 30% w/w to 31% w/w of the total weight of the composition. In some embodiments, the weight percent of the particles may be from 30% w/w to 40%, 35% w/w to 40%, 38% w/w to 42%, or 39% w/w to 40%. In some embodiments, the weight percent of the particles may be 40% w/w of the total weight of the composition.

Octreotide is an octapeptide. The octreotide may be in free form, salt form or in the form of complexes thereof. In some examples, acid addition salts may be formed with organic acids, polymeric acids, and/or inorganic acids. Acid addition salts may include, for example, hydrochloride and acetates. Complexes may be formed from octreotide on addition of inorganic substances, e.g., inorganic salts or hydroxides such as Ca- and Zn-salts and/or addition of polymeric organic substances. The octreotide may include a pharmaceutically acceptable salt. In some embodiments, the pharmaceutically acceptable salt is acetate.

The weight percent of the octreotide or a pharmaceutically acceptable salt of octreotide in the composition may be from 1% w/w to 30% w/w of the total weight of the composition. In various embodiments, the weight percent of the octreotide may be from about 1% w/w to about 30% w/w, about 1% w/w to about 25% w/w, about 1% w/w to about 20% w/w, about 1% w/w to about 15%, about 1% w/w to about 10% w/w, about 1% w/w to about 5% w/w, about 1.25% w/w to about 5% w/w, about 1.5% w/w to about 5% w/w, about 1.75% w/w to about 5% w/w, about 2% w/w to about 5% w/w, about 2.25% w/w to about 5% w/w, about 2.5% w/w to about 5% w/w, about 2.75% w/w to about 5% w/w, about 3% w/w to about 5% w/w, about 3.25% w/w to about 5% w/w, about 3.5% w/w to about 5% w/w, about 3.75% w/w to about 5% w/w, about 4% w/w to about 5% w/w, about 4.25% w/w to about 5% w/w, about 4.5% w/w to about 5% w/w, or about 4.75% w/w to about 5% w/w. The weight percent of the octreotide may be from about 3% w/w to about 4% w/w, about 3.1% w/w to about 3.9% w/w, about 3.2% w/w to about 3.8% w/w, about 3.3% w/w to about 3.7% w/w, about 3.4% w/w to about 3.6% w/w, about 3.3% w/w to about 3.6% w/w, or about 3.3% w/w to about 3.5% w/w. In some embodiments, the weight percent of the octreotide may be about 3.5% w/w of the total weight of the composition.

In some embodiments, the weight percent of the octreotide may be from 1% w/w to 30% w/w, 1% w/w to 25% w/w, 1% w/w to 20% w/w, 1% w/w to 15%, 1% w/w to 10% w/w, 1% w/w to 5% w/w, 1.25% w/w to 5% w/w, 1.5% w/w to 5% w/w, 1.75% w/w to 5% w/w, about 2% w/w to 5% w/w, 2.25% w/w to 5% w/w, 2.5% w/w to 5% w/w, 2.75% w/w to 5% w/w, 3% w/w to 5% w/w, 3.25% w/w to 5% w/w, 3.5% w/w to 5% w/w, 3.75% w/w to 5% w/w, 4% w/w to 5% w/w, 4.25% w/w to 5% w/w, 4.5% w/w to 5% w/w, or about 4.75% w/w to about 5% w/w. In other embodiments, the weight percent of the octreotide may be from 3% w/w to 4% w/w, 3.1% w/w to 3.9% w/w, 3.2% w/w to 3.8% w/w, 3.3% w/w to 3.7% w/w, 3.4% w/w to 3.6% w/w, 3.3% w/w to 3.6% w/w, or 3.3% w/w to 3.5% w/w. In some embodiments, the weight percent of the octreotide may be 3.5% w/w of the total weight of the composition.

The composition may include one or more peptides. In some embodiments, the one or more peptides may comprise octreotide (8 amino acids) leuprolide (9 amino acids), oxytocin (9 amino acids), goserelin (10 amino acids), triptorelin (10 amino acids), buserelin (9 amino acids), deslorelin (9 amino acids), vasopressin (9 amino acids), lypressin (8 amino acids), somatostatin (14 amino acids), melanocyte-stimulating hormone (MSH, 13 amino acids), angiotensin II (8 amino acids), or terlipressin (12 amino acids).

The sequences of the peptides and their sequence identification numbers are provided in Table 1.

TABLE 1
List of peptides sequences and their sequence identification numbers.

| | | | | Sequence |
|---|---|---|---|---|
| | | | | identi- |
| | | | | fication |
| | | | Amino acid | number |
| Peptide | Sequence as IUPAC name | IUPAC condensed | sequence | (SEQ ID) |
| Octreotide | D-phenylalanyl-L-cysteinyl-L- | H-D-Phe-Cys(1)- | FCFWKTCT | SEQ ID NO: 1 |
| | phenylalanyl-D-tryptophyl-L-lysyl- | Phe-D-Trp-Lys- | | |
| | L-threonyl-L-cysteinyl-L- | Thr-Cys(1)-Thr-ol | | |
| | threoninol (2→7)-disulfide | | | |
| Leuprolide | L-pyroglutamyl-L-histidyl-L- | H-Pyr-His-Trp- | XHWSYLLRP | SEQ ID NO: 2 |
| | tryptophyl-L-seryl-L-tyrosyl-D- | Ser-Tyr-D-Leu- | | |
| | leucyl-L-leucyl-L-arginyl-L- | Leu-Arg-Pro-NHEt | | |
| | proline ethylamide | | | |
| Oxytocin | L-cysteinyl-L-tyrosyl-L-isoleucyl- | H-Cys(1)-Tyr-Ile- | CYIQNCPLG | SEQ ID NO: 3 |
| | L-glutaminyl-L-asparagyl-L- | Gln-Asn-Cys(1)- | | |
| | cysteinyl-L-prolyl-L-leucyl- | Pro-Leu-Gly-NH2 | | |
| | glycinamide (1→6)-disulfide | | | |
| Goserelin | L-pyroglutamyl-L-histidyl-L- | H-Pyr-His-Trp- | XHWSYXLRP | SEQ ID NO: 4 |
| | tryptophyl-L-seryl-L-tyrosyl-O- | Ser-Tyr-D- | | |
| | tert-butyl-D-seryl-L-leucyl-L- | Ser(tBu)-Leu-Arg- | | |
| | arginyl-N′-carbamoyl-L- | Pro- | | |
| | prolinehydrazide | NHNHCONH2 | | |
| Triptorelin | L-pyroglutamyl-L-histidyl-L- | H-Pyr-His-Trp- | XHWSYWLRPG | SEQ ID NO: 5 |
| | tryptophyl-L-seryl-L-tyrosyl-D- | Ser-Tyr-D-Trp- | | |
| | tryptophyl-L-leucyl-L-arginyl-L- | Leu-Arg-Pro-Gly- | | |
| | prolyl-glycinamide | NH2 | | |
| Buserelin | L-pyroglutamyl-L-histidyl-L- | H-Pyr-His-Trp- | XHWSYXLRP | SEQ ID NO: 6 |
| | tryptophyl-L-seryl-L-tyrosyl-O- | Ser-Tyr-D- | | |
| | tert-butyl-D-seryl-L-leucyl-L- | Ser(tBu)-Leu-Arg- | | |
| | arginyl-L-proline ethylamide | Pro-NHEt | | |
| Deslorelin | L-pyroglutamyl-L-histidyl-L- | H-Pyr-His-Trp- | XHWSYWLRP | SEQ ID NO: 7 |
| | tryptophyl-L-seryl-L-tyrosyl-O- | Ser-Tyr-D-Trp- | | |
| | tert-butyl-D-seryl-L-leucyl-L- | Leu-Arg-Pro-NHEt | | |
| | arginyl-L-proline ethylamide | | | |
| Vasopressin | DL-cysteinyl-DL-tyrosyl-DL- | H-DL-Cys(1)-DL- | CYFQNCPKG | SEQ ID NO: 8 |
| | phenylalanyl-DL-glutaminyl-DL- | Tyr-DL-Phe-DL- | | |
| | asparagyl-DL-cysteinyl-DL-prolyl- | Gln-DL-Asn-DL- | | |
| | DL-lysyl-glycinamide (1→6)- | Cys(1)-DL-Pro- | | |
| | disulfidecompound with DL- | DL-Lys-Gly- | | |
| | cysteinyl-DL-tyrosyl-DL- | NH2.H-DL- | | |
| | phenylalanyl-DL-glutaminyl-DL- | Cys(2)-DL-Tyr- | | |
| | asparagyl-DL-cysteinyl-DL-prolyl- | DL-Phe-DL-Gln- | | |
| | DL-arginyl-glycinamide (1′→6′)- | DL-Asn-DL- | | |
| | disulfide | Cys(2)-DL-Pro- | | |
| | | DL-Arg-Gly-NH2 | | |
| Lypressin | L-cysteinyl-L-tyrosyl-L- | H-Cys(1)-Tyr-Phe- | CYFQNCPKG | SEQ ID NO: 9 |
| | phenylalanyl-L-glutaminyl-L- | Gln-Asn-Cys(1)- | | |
| | asparagyl-L-cysteinyl-L-prolyl-L- | Pro-Lys-Gly-NH2 | | |
| | lysyl-glycinamide (1→6)-disulfide | | | |
| Somatostatin | L-alanyl-glycyl-L-cysteinyl-L- | H-Ala-Gly-Cys(1)- | AGCKNFFWKT | SEQ ID NO: 10 |
| | lysyl-L-asparagyl-L-phenylalanyl- | Lys-Asn-Phe-Phe- | FTSC | |
| | L-phenylalanyl-L-tryptophyl-L- | Trp-Lys-Thr-Phe- | | |
| | lysyl-L-threonyl-L-phenylalanyl-L- | Thr-Ser-Cys(1)- | | |
| | threonyl-L-seryl-L-cysteine (3→4)- | OH | | |
| | disulfide | | | |
| Melanocyte- | N-acetyl-L-seryl-L-tyrosyl-L- | Ac-Ser-Tyr-Ser- | SYSMEHFRWG | SEQ ID NO: 11 |
| Stimulating | seryl-L-methionyl-L-alpha- | Met-Glu-His-Phe- | KPV | |
| Hormone | glutamyl-L-histidyl-L- | Arg-Trp-Gly-Lys- | | |
| | phenylalanyl-L-arginyl-L- | Pro-Val-NH2 | | |
| | tryptophyl-glycyl-L-lysyl-L- | | | |
| | prolyl-L-valinamide | | | |
| Angiotensin | L-alpha-aspartyl-L-arginyl-L- | H-Asp-Arg-Val- | DRVYIHPF | SEQ ID NO: 12 |
| II | valyl-L-tyrosyl-L-isoleucyl-L- | Tyr-Ile-His-Pro- | | |
| | histidyl-L-prolyl-L-phenylalanine | Phe-OH | | |
| Terlipressin | glycyl-glycyl-glycyl-L-cysteinyl- | H-Gly-Gly-Gly- | GGGCYFQNCP | SEQ ID NO: 13 |
| | L-tyrosyl-L-phenylalanyl-L- | Cys(1)-Tyr-Phe- | KG | |
| | glutaminyl-L-asparagyl-L- | Gln-Asn-Cys(1)- | | |
| | cysteinyl-L-prolyl-L-lysyl- | Pro-Lys-Gly-NH2 | | |
| | glycinamide (4→9)-disulfide | | | |

The permeability enhancer may be a fatty acid or fatty acid derivative, a surfactant, a stabilizing agent, a chemical modifier, or an enzyme inhibitor. Examples of the fatty acid or fatty acid derivative include but are not limited to sodium caprylate, oleic acid, or lauric acid. Examples of the surfactants include but are not limited to sodium lauryl sulfate (SLS), polysorbates, or Triton X-100. Examples of chemical modifiers include but are not limited to cyclodextrins, azone, or menthol. Examples of enzyme inhibitors include but are not limited to protease inhibitors or P-Glycoprotein inhibitors. In some embodiments, the permeability enhancer may comprise sodium caprylate. In some embodiments, the permeability enhancer may comprise sodium caprylate, polysorbate 80, ethylenediaminetetraacetic acid (EDTA), or any combination thereof.

In some embodiments, the weight percent of the permeability enhancers in the composition may be from 0.1% w/w to 20% w/w of the total weight of the composition. In various embodiments, the weight percent of the permeability enhancers may be from about 0.1% w/w to about 20% w/w, about 0.5% w/w to about 20% w/w, about 0.5% w/w to about 19% w/w, about 0.5% w/w to about 18% w/w, about 0.5% w/w to about 17% w/w, about 0.5% w/w to about 16% w/w, about 0.5% w/w to about 15% w/w, about 0.5% w/w to about 14% w/w, about 0.5% w/w to about 13% w/w, about 0.5% w/w to about 12% w/w, about 0.5% w/w to about 11% w/w, about 0.5% w/w to about 10% w/w, about 0.6% w/w to about 12% w/w, about 0.6% w/w to about 11% w/w, about 0.7% w/w to about 10% w/w, about 0.8% w/w to about 10% w/w, about 8.2% w/w to about 9.8% w/w, about 8.4% w/w to about 9.6% w/w, about 8.6% w/w to about 9.4% w/w, or about 8.8% w/w to about 9.2% w/w. In some embodiments, the weight percent of the permeability enhancers may be about 9% w/w of the total weight of the composition.

In some embodiments, the weight percent of the permeability enhancers may be from 1% w/w to 20% w/w, 5% w/w to 20% w/w, 5% w/w to 19% w/w, 5% w/w to 18% w/w, 5% w/w to 17% w/w, 5% w/w to 16% w/w, 5% w/w to 15% w/w, 5% w/w to 14% w/w, 5% w/w to 13% w/w, 5% w/w to 12% w/w, 5% w/w to 11% w/w, 5% w/w to 10% w/w, 6% w/w to 12% w/w, 6% w/w to 11% w/w, 7% w/w to 10% w/w, 8% w/w to 10% w/w, 8.2% w/w to 9.8% w/w, 8.4% w/w to 9.6% w/w, 8.6% w/w to 9.4% w/w, or 8.8% w/w to 9.2% w/w. In some embodiments, the weight percent of the permeability enhancers may be 9% w/w of the total weight of the composition.

In various embodiments, the composition may comprise a stabilizing agent. In various embodiments, the stabilizing agents may comprise any suitable stabilizing agent for example, chelating compounds EDTA, deferoxamine, dimercaprol (British Anti-Lewisite), penicillamine, succimer (dimercaptosuccinic Acid, DMSA), thiamine monophosphate (thiamine chelate), trientine, calcium disodium EDTA, ferric citrate, or any other suitable stabilizing agent. In some embodiments, the stabilizing agents may comprise EDTA. The presence of stabilizing agents may result in chelating minerals.

In some embodiments, the weight percent of the stabilizing agent in the composition may comprise 0.1% w/w to 15% w/w, 0.1% w/w to 5% w/w, or 0.1% w/w to 2% w/w of the total weight of the composition. In various embodiments, the weight percent of the stabilizing agent may be from about 0.1% w/w to about 15% w/w, about 0.1% w/w to about 5% w/w, about 0.1% w/w to about 2% w/w, about 0.1% w/w to about 1% w/w, about 0.1% w/w to about 1.9% w/w, about 0.1% w/w to about 1.8% w/w, about 0.1% w/w to about 1.7% w/w, about 0.1% w/w to about 1.6% w/w, about 0.1% w/w to about 1.5% w/w, about 0.1% w/w to about 1.4% w/w, about 0.1% w/w to about 1.3% w/w, about 0.1% w/w to about 1.2% w/w, about 0.1% w/w to about 1.1% w/w, about 0.1% w/w to about 1.0% w/w, about 0.1% w/w to about 0.9% w/w, about 0.1% w/w to about 0.8% w/w, about 0.1% w/w to about 0.7% w/w, about 0.1% w/w to about 0.6% w/w, about 0.1% w/w to about 0.5% w/w, about 0.1% w/w to about 0.4% w/w, about 0.1% w/w to about 0.3% w/w, or about 0.1% w/w to about 0.2% w/w. The weight percent of the stabilizing agent may be from about 0.1% w/w to about 1% w/w, about 0.2% w/w to about 0.9% w/w, about 0.3% w/w to about 0.8% w/w, about 0.3% w/w to about 0.7% w/w, about 0.3% w/w to about 0.6% w/w, about 0.3% w/w to about 0.5% w/w, about 0.3% w/w to about 0.4% w/w, about 0.4% w/w to about 0.7% w/w, or about 0.5% w/w to about 0.6% w/w. In some embodiments, the weight percent of the stabilizing agent may be about 0.4% w/w of the total weight of the composition.

In some embodiments, the weight percent of the stabilizing agent may be from 0.1% w/w to 15% w/w, 0.1% w/w to 5% w/w, 0.1% w/w to 1% w/w, 0.1% w/w to 2% w/w, 0.1% w/w to 1.9% w/w, 0.1% w/w to 1.8% w/w, 0.1% w/w to 1.7% w/w, 0.1% w/w to 1.6% w/w, 0.1% w/w to 1.5% w/w, 0.1% w/w to 1.4% w/w, 0.1% w/w to 1.3% w/w, 0.1% w/w to 1.2% w/w, 0.1% w/w to 1.1% w/w, 0.1% w/w to 1.0% w/w, 0.1% w/w to 0.9% w/w, 0.1% w/w to 0.8% w/w, 0.1% w/w to 0.7% w/w, 0.1% w/w to 0.6% w/w, 0.1% w/w to 0.5% w/w, 0.1% w/w to 0.4% w/w, 0.1% w/w to 0.3% w/w, or 0.1% w/w to 0.2% w/w. The weight percent of the stabilizing agent may be from 0.1% w/w to 1% w/w, 0.2% w/w to 0.9% w/w, 0.3% w/w to 0.8% w/w, 0.3% w/w to 0.7% w/w, 0.3% w/w to 0.6% w/w, 0.3% w/w to 0.5% w/w, 0.3% w/w to 0.4% w/w, 0.4% w/w to 0.7% w/w, or 0.5% w/w to 0.6% w/w. In some embodiments, the weight percent of the stabilizing agent may be 0.4% w/w of the total weight of the composition.

In some embodiments, the composition may comprise a stabilizing agent. In some examples, the stabilizing agent comprises at least one amino acid. In some embodiments, the amino acid may comprise leucine, cysteine, tryptophan, methionine, lysine, arginine, tyrosine, glycine, histidine or any combination thereof. In some embodiments, the amino acid may comprise leucine. In additional embodiments, the composition may comprise more than one amino acid. In some embodiments, the composition may comprise leucine and phenylalanine. In various embodiments the composition may include phenylalanine and glutamine. In yet another embodiment, the composition may comprise leucine, phenylalanine, and glutamine.

The weight percent of the amino acid in the composition may be from 0.01% w/w to 5% w/w of the total weight of the composition. In various embodiments, the weight percent of amino acid may be from about 0.01% w/w to about 5% w/w, about 0.01% to about 2.5% w/w, about 0.01% to about 1% w/w, 0.01% w/w to about 0.5% w/w, about 0.02% w/w to about 0.5% w/w, about 0.03% w/w to about 0.5% w/w, about 0.04% w/w to about 0.5% w/w, about 0.05% w/w to about 0.5% w/w, about 0.06% w/w to about 0.5% w/w, about 0.07% w/w to about 0.5% w/w, about 0.08% w/w to about 0.5% w/w, about 0.09% w/w to about 0.5% w/w, about 0.10% w/w to about 0.5% w/w, about 0.11% w/w to about 0.5% w/w, about 0.12% w/w to about 0.5% w/w, about 0.13% w/w to about 0.5% w/w, about 0.14% w/w to about 0.5% w/w, about 0.15% w/w to about 0.5% w/w, about 0.16% w/w to about 0.5% w/w, about 0.17% w/w to about 0.5% w/w, about 0.18% w/w to about 0.5% w/w, about 0.19% w/w to about 0.5% w/w, about 0.20% w/w to about 0.5% w/w, about 0.01% w/w to about 0.45% w/w, about 0.05% w/w to about 0.4% w/w, about 0.1% w/w to about 0.35% w/w, about 0.11% w/w to about 0.34% w/w, about 0.12% w/w to about 0.33% w/w, about 0.13% w/w to about 0.32% w/w, about 0.14% w/w to about 0.31% w/w, about 0.15% w/w to about 0.30% w/w, about 0.16% w/w to about 0.25% w/w, about 0.01% w/w to about 0.05% w/w, about 0.05% w/w to about 0.1% w/w, about 0.1% w/w to about 0.15% w/w, about 0.15% w/w to about 0.2% w/w, about 0.2% w/w to about 0.25% w/w, about 0.25% w/w to about 0.3% w/w, about 0.3% w/w to about 0.35% w/w, about 0.4% w/w to about 0.45% w/w, or about 0.45% w/w to about 0.5% w/w. The weight percent of amino acid may be from about 0.1% w/w to about 0.4% w/w, about 0.12% w/w to about 0.38% w/w, about 0.14% w/w to about 0.36% w/w, about 0.16% w/w to about 0.34% w/w, about 0.18% w/w to about 0.32% w/w, about 0.2% w/w to about 0.3% w/w, or about 0.14% w/w to about 0.18% w/w. In some embodiments, the weight percent of amino acid may be about 0.16% w/w of the total weight of the composition.

In some embodiments, the weight percent of amino acid may be from 0.01% w/w to 5% w/w, 0.01% to 2.5% w/w, 0.01% to 1% w/w, 0.01% w/w to 0.5% w/w, 0.02% w/w to 0.5% w/w, 0.03% w/w to 0.5% w/w, 0.04% w/w to 0.5% w/w, 0.05% w/w to 0.5% w/w, 0.06% w/w to 0.5% w/w, 0.07% w/w to 0.5% w/w, 0.08% w/w to 0.5% w/w, 0.09% w/w to 0.5% w/w, 0.10% w/w to 0.5% w/w, 0.11% w/w to 0.5% w/w, 0.12% w/w to 0.5% w/w, 0.13% w/w to 0.5% w/w, 0.14% w/w to 0.5% w/w, 0.15% w/w to 0.5% w/w, 0.16% w/w to 0.5% w/w, 0.17% w/w to 0.5% w/w, 0.18% w/w to 0.5% w/w, 0.19% w/w to 0.5% w/w, 0.20% w/w to 0.5% w/w, 0.01% w/w to 0.45% w/w, 0.05% w/w to 0.4% w/w, 0.1% w/w to 0.35% w/w, 0.11% w/w to 0.34% w/w, 0.12% w/w to 0.33% w/w, 0.13% w/w to 0.32% w/w, 0.14% w/w to 0.31% w/w, 0.15% w/w to 0.30% w/w, 0.16% w/w to 0.25% w/w, 0.01% w/w to 0.05% w/w, 0.05% w/w to 0.1% w/w, 0.1% w/w to 0.15% w/w, 0.15% w/w to 0.2% w/w, 0.2% w/w to 0.25% w/w, 0.25% w/w to 0.3% w/w, 0.3% w/w to 0.35% w/w, 0.4% w/w to 0.45% w/w, or 0.45% w/w to 0.5% w/w. The weight percent of amino acid may be from 0.1% w/w to 0.4% w/w, 0.12% w/w to 0.38% w/w, 0.14% w/w to 0.36% w/w, 0.16% w/w to 0.34% w/w, 0.18% w/w to 0.32% w/w, 0.2% w/w to 0.3% w/w, or 0.14% w/w to 0.18% w/w. In some embodiments, the weight percent of amino acid may be 0.16% w/w of the total weight of the composition.

Addition of amino acids in the composition may significantly reduce potential peptide impurities. The presence of impurities in the composition may be characterized using high-performance liquid chromatography (HPLC). In some embodiments, delayed release compositions comprising at least one amino acid may have less impurities detected by HPLC compared to delayed release compositions not comprising an amino acid. Impurities may include but are not limited to acetyl octreotide. In some embodiments, the method for measuring impurities may include reverse phase HPLC using a temperature of 30-50 °C and a mobile phase comprising a mobile phase A of about 0.02% trifluoroacetic acid (TFA) in water and a mobile phase B of 100% acetonitrile. In alternative embodiments, any other suitable HPLC methods may be used.

In some examples, the composition may have reduced impurities compared to other delayed release octreotide compositions. The other delayed release octreotide compositions may include oil dispersions (e.g. sold under the trademark Mycapssa®) as well as other solid dosage compositions that are not oil dispersions.

In some embodiments, the composition may have an amount of total impurities of less than 10%, less than 5%, less than 2%, less than 1% w/w, less than 0.95% w/w, less than 0.9% w/w, less than 0.85% w/w, less than 0.8% w/w, less than 0.75% w/w, less than 0.7% w/w, less than 0.65% w/w, less than 0.6% w/w, less than 0.55% w/w, less than 0.5% w/w, less than 0.45% w/w, less than 0.4% w/w, less than 0.35% w/w, less than 0.3% w/w, less than 0.25% w/w, less than 0.2% w/w, less than 0.15% w/w, less than 0.1% w/w, or less than 0.05% w/w.

In some embodiments, the composition may have an amount of acetyl-octreotide of less than 10%, less than 5%, less than 2%, less than 1% w/w, less than 0.95% w/w, less than 0.9% w/w, less than 0.85% w/w, less than 0.8% w/w, less than 0.75% w/w, less than 0.7% w/w, less than 0.65% w/w, less than 0.6% w/w, less than 0.55% w/w, less than 0.5% w/w, less than 0.45% w/w, less than 0.4% w/w, less than 0.35% w/w, less than 0.3% w/w, less than 0.25% w/w, less than 0.2% w/w, less than 0.15% w/w, less than 0.1% w/w, less than 0.05% w/w, or less than 0.01% w/w.

In various embodiments, the delayed release compositions as described comprising an amino acid may have a reduced total impurity % compared to compositions without an amino acid. In some embodiments, the delayed release compositions may have a similar amount of total impurities compared to the approved delayed release oral composition of octreotide sold under the trademark Mycapssa®.

The ratio of octreotide or salt thereof to amino acid may be from about 1:1 to about 50:1 on a w/w basis. In various embodiments the ratio may be 1:1 to 50:1, 2:1 to 50:1, 3:1 to 50:1, 4:1 to 50:1, 5:1 to 50:1, 6:1 to 50:1, 7:1 to 50:1, 8:1 to 50:1, 9:1 to 50:1, 10:1 to 50:1, 11:1 to 50:1, 12:1 to 50:1, 13:1 to 50:1, 14:1 to 50:1, 15:1 to 50:1, 16:1 to 50:1, 17:1 to 50:1, 18:1 to 50:1, 19:1 to 50:1, 20:1 to 50:1, 21:1 to 50:1, 22:1 to 50:1, 23:1 to 50:1, 24:1 to 50:1, 25:1 to 50:1, 26:1 to 50:1, 27:1 to 50:1, 28:1 to 50:1, 29:1 to 50:1, 30:1 to 50:1, 31:1 to 50:1, 32:1 to 50:1, 33:1 to 50:1, 34:1 to 50:1, 35:1 to 50:1, 36:1 to 50:1, 37:1 to 50:1, 38:1 to 50:1, 39:1 to 50:1, 40:1 to 50:1, 41:1 to 50:1, 42:1 to 50:1, 43:1 to 50:1, 44:1 to 50:1, 45:1 to 50:1, 46:1 to 50:1, 47:1 to 50:1, 48:1 to 50:1, 49:1 to 50:1 on a w/w basis. In some embodiments, the ratio may be from 10:1 to 40:1, 15:1 to 35:1, or 20:1 to 30:1 on a w/w basis. In some embodiments, the ratio may be 22:1 on a w/w basis.

The divalent salts may comprise salts of calcium, magnesium, zinc, or iron. Examples of calcium salts include but are not limited to calcium carbonate, calcium citrate, or calcium chloride. Examples of magnesium salts include but are not limited to magnesium sulfate, magnesium stearate, or magnesium chloride. Examples of zinc salts include but are not limited to zinc sulfate, zinc acetate, or zinc chloride. In some embodiments, the divalent salt may be magnesium chloride.

The weight percent of the divalent salt in the composition may be from 0.01% w/w to 1% w/w of the total weight of the composition. In various embodiments, the weight percent of the divalent salt may be from about 0.01% w/w to about 1% w/w, about 0.02% w/w to about 1% w/w, about 0.03% w/w to about 1% w/w, about 0.04% w/w to about 1% w/w, about 0.05% w/w to about 1% w/w, about 0.06% w/w to about 1% w/w, about 0.07% w/w to about 1% w/w, about 0.08% w/w to about 1% w/w, about 0.09% w/w to about 1% w/w, about 0.10% w/w to about 1% w/w, about 0.11% w/w to about 1% w/w, about 0.12% w/w to about 1% w/w, about 0.13% w/w to about 1% w/w, about 0.14% w/w to about 1% w/w, about 0.15% w/w to about 1% w/w, about 0.16% w/w to about 1% w/w, about 0.17% w/w to about 1% w/w, about 0.18% w/w to about 1% w/w, about 0.19% w/w to about 1% w/w, about 0.20% w/w to about 1% w/w, about 0.21% w/w to about 1% w/w, about 0.22% w/w to about 1% w/w, about 0.23% w/w to about 1% w/w, about 0.24% w/w to about 1% w/w, about 0.25% w/w to about 1% w/w, about 0.26% w/w to about 1% w/w, about 0.27% w/w to about 1% w/w, about 0.28% w/w to about 1% w/w, about 0.29% w/w to about 1% w/w, about 0.30% w/w to about 1% w/w, about 0.31% w/w to about 1% w/w, about 0.32% w/w to about 1% w/w, about 0.33% w/w to about 1% w/w, about 0.34% w/w to about 1% w/w, about 0.35% w/w to about 1% w/w, about 0.36% w/w to about 1% w/w, about 0.37% w/w to about 1% w/w, about 0.38% w/w to about 1% w/w, about 0.39% w/w to about 1% w/w, about 0.40% w/w to about 1% w/w, about 0.41% w/w to about 1% w/w, about 0.42% w/w to about 1% w/w, about 0.43% w/w to about 1% w/w, about 0.44% w/w to about 1% w/w, about 0.45% w/w to about 1% w/w, about 0.46% w/w to about 1% w/w, about 0.47% w/w to about 1% w/w, about 0.48% w/w to about 1% w/w, about 0.49% w/w to about 1% w/w, about 0.01% w/w to about 0.5% w/w, about 0.02% w/w to about 0.5% w/w, about 0.03% w/w to about 0.5% w/w, about 0.04% w/w to about 0.5% w/w, about 0.05% w/w to about 0.5% w/w, about 0.06% w/w to about 0.5% w/w, about 0.07% w/w to about 0.5% w/w, about 0.08% w/w to about 0.5% w/w, about 0.09% w/w to about 0.5% w/w, about 0.10% w/w to about 0.5% w/w, about 0.1% w/w to about 1% w/w, about 0.11% w/w to about 0.5% w/w, about 0.12% w/w to about 0.5% w/w, about 0.13% w/w to about 0.5% w/w, about 0.14% w/w to about 0.5% w/w, about 0.15% w/w to about 0.5% w/w, about 0.16% w/w to about 0.5% w/w, about 0.17% w/w to about 0.5% w/w, about 0.18% w/w to about 0.5% w/w, about 0.19% w/w to about 0.5% w/w, about 0.20% w/w to about 0.5% w/w, about 0.21% w/w to about 0.5% w/w, about 0.22% w/w to about 0.5% w/w, about 0.23% w/w to about 0.5% w/w, about 0.24% w/w to about 0.5% w/w, about 0.25% w/w to about 0.5% w/w, about 0.26% w/w to about 0.5% w/w, about 0.27% w/w to about 0.5% w/w, about 0.28% w/w to about 0.5% w/w, about 0.29% w/w to about 0.5% w/w, about 0.30% w/w to about 0.5% w/w, about 0.31% w/w to about 0.5% w/w, about 0.32% w/w to about 0.5% w/w, about 0.33% w/w to about 0.5% w/w, about 0.34% w/w to about 0.5% w/w, about 0.35% w/w to about 0.5% w/w, about 0.36% w/w to about 0.5% w/w, about 0.37% w/w to about 0.5% w/w, about 0.38% w/w to about 0.5% w/w, about 0.39% w/w to about 0.5% w/w, about 0.40% w/w to about 0.5% w/w, about 0.41% w/w to about 0.5% w/w, about 0.42% w/w to about 0.5% w/w, about 0.43% w/w to about 0.5% w/w, about 0.44% w/w to about 0.5% w/w, about 0.45% w/w to about 0.5% w/w, about 0.46% w/w to about 0.5% w/w, about 0.47% w/w to about 0.5% w/w, about 0.48% w/w to about 0.5% w/w, or about 0.49% w/w to about 0.5% w/w. The weight percent of the divalent salt may be from about 0.1% w/w to about 0.4% w/w, about 0.12% w/w to about 0.38% w/w, about 0.14% w/w to about 0.36% w/w, about 0.16% w/w to about 0.34% w/w, about 0.18% w/w to about 0.32% w/w, about 0.2% w/w to about 0.3% w/w, or about 0.14% w/w to about 0.18% w/w. In some embodiments, the weight percent may be 0.1% w/w to about 1% w/w. In some embodiments, the weight percent of the divalent salt may be about 0.16% w/w of the total weight of the composition.

In some embodiments, the weight percent of the divalent salt may be from 0.01% w/w to 0.5% w/w, 0.02% w/w to 0.5% w/w, 0.03% w/w to 0.5% w/w, 0.04% w/w to 0.5% w/w, 0.05% w/w to 0.5% w/w, 0.06% w/w to 0.5% w/w, 0.07% w/w to 0.5% w/w, 0.08% w/w to 0.5% w/w, 0.09% w/w to 0.5% w/w, 0.10% w/w to 0.5% w/w, 0.1% w/w to 1% w/w, 0.11% w/w to 0.5% w/w, 0.12% w/w to 0.5% w/w, 0.13% w/w to 0.5% w/w, 0.14% w/w to 0.5% w/w, 0.15% w/w to 0.5% w/w, 0.16% w/w to 0.5% w/w, 0.17% w/w to 0.5% w/w, 0.18% w/w to 0.5% w/w, 0.19% w/w to 0.5% w/w, 0.20% w/w to 0.5% w/w, 0.21% w/w to 0.5% w/w, 0.22% w/w to 0.5% w/w, 0.23% w/w to 0.5% w/w, 0.24% w/w to 0.5% w/w, 0.25% w/w to 0.5% w/w, 0.26% w/w to 0.5% w/w, 0.27% w/w to 0.5% w/w, 0.28% w/w to 0.5% w/w, 0.29% w/w to 0.5% w/w, 0.30% w/w to 0.5% w/w, 0.31% w/w to 0.5% w/w, 0.32% w/w to 0.5% w/w, 0.33% w/w to 0.5% w/w, 0.34% w/w to 0.5% w/w, 0.35% w/w to 0.5% w/w, 0.36% w/w to 0.5% w/w, 0.37% w/w to 0.5% w/w, 0.38% w/w to 0.5% w/w, 0.39% w/w to 0.5% w/w, 0.40% w/w to 0.5% w/w, 0.41% w/w to 0.5% w/w, 0.42% w/w to 0.5% w/w, 0.43% w/w to 0.5% w/w, 0.44% w/w to 0.5% w/w, 0.45% w/w to 0.5% w/w, 0.46% w/w to 0.5% w/w, 0.47% w/w to 0.5% w/w, 0.48% w/w to 0.5% w/w, or 0.49% w/w to 0.5% w/w. The weight percent of the divalent salt may be from 0.1% w/w to 0.4% w/w, 0.12% w/w to 0.38% w/w, 0.14% w/w to 0.36% w/w, 0.16% w/w to 0.34% w/w, 0.18% w/w to 0.32% w/w, 0.2% w/w to 0.3% w/w, or 0.14% w/w to 0.18% w/w. In some embodiments, the weight percent may be 0.1% w/w to 1% w/w. In some embodiments, the weight percent of the divalent salt may be 0.16% w/w of the total weight of the composition.

In some embodiments, the binder may comprise a natural binder, a synthetic binder, a synthetic polymer, or other materials. Natural binders include but are not limited to starch and its derivatives, cellulose (e.g. microcrystalline cellulose), gelatin, or acacia and guar gum. Synthetic binders include but are not limited to polyvinylpyrrolidone (PVP), hydroxypropyl methylcellulose (HPMC), or polyethylene glycol (PEG). Synthetic polymers include but are not limited to ethylcellulose or polyvinyl alcohol (PVA). Other binders include magnesium stearate or lactose. In some embodiments, the binder may comprise polyvinylpyrrolidone (PVP). In various embodiments, the binder may comprise more than one form of PVP. In additional embodiments the PVP may comprise PVP-12, PVP-17 (Povidone K17), PVP K-90, and PVP K-30. In some embodiments, the binder may comprise PVP-12, PVP-17, or both.

The weight percent of the binder in the composition may be from 0.1% w/w to 10% w/w of the total weight of the composition. In various embodiments, the weight percent of the binder may be from about 0.1% w/w to about 10% w/w, about 1% w/w to about 10% w/w, about 1% w/w to about 7% w/w, about 1.5% w/w to about 7% w/w, about 2% w/w to about 7% w/w, about 2.5% w/w to about 7% w/w, about 3% w/w to about 7% w/w, about 3.5% w/w to about 7% w/w, about 4% w/w to about 7% w/w, about 4.5% w/w to about 7% w/w, about 5% w/w to about 7% w/w, about 5.5% w/w to about 7% w/w, about 6% w/w to about 7% w/w, or about 6.5% w/w to about 7% w/w.

In some embodiments, the weight percent of the binder may be from 0.11% to 10%, 1% w/w to 10% w/w, 1% w/w to 7% w/w, 1.5% w/w to 7% w/w, 2% w/w to 7% w/w, 2.5% w/w to 7% w/w, 3% w/w to 7% w/w, 3.5% w/w to 7% w/w, 4% w/w to 7% w/w, 4.5% w/w to 7% w/w, 5% w/w to 7% w/w, 5.5% w/w to 7% w/w, 6% w/w to 7% w/w, or 6.5% w/w to 7% w/w.

The weight percent of the binder may be from about 1.5% w/w to about 2.5% w/w, about 1.6% w/w to about 2.4% w/w, about 1.7% w/w to about 2.3% w/w, about 1.8% w/w to about 2.2% w/w, about 1.9% w/w to about 2.0% w/w, about 1.85% w/w to about 2.05% w/w, or about 1.9% w/w to about 2.1% w/w. The weight percent of the binder may be from about 2.5% w/w to about 5.5% w/w, about 2.6% w/w to about 5.4% w/w, about 2.7% w/w to about 5.3% w/w, about 2.8% w/w to about 5.2% w/w, about 2.9% w/w to about 5.1% w/w, about 3.0% w/w to about 5.0% w/w, about 3.1% w/w to about 4.9% w/w, about 3.2% w/w to about 4.8% w/w, about 3.3% w/w to about 4.7% w/w, about 3.4% w/w to about 4.6% w/w, about 3.5% w/w to about 4.5% w/w, about 3.6% w/w to about 4.4% w/w, about 3.7% w/w to about 4.3% w/w, about 3.8% w/w to about 4.2% w/w, or about 3.9% w/w to about 4.1% w/w.

In some embodiments, the weight percent of the binder may be about 1% w/w to about 10% w/w. In some embodiments, the weight percent of the binder may be about 1% w/w to about 5% w/w. In some embodiments, the weight percent of the binder may be about 6% w/w of the total weight of the composition.

The weight percent of the binder may be from 1.5% w/w to 2.5% w/w, 1.6% w/w to 2.4% w/w, 1.7% w/w to 2.3% w/w, 1.8% w/w to 2.2% w/w, 1.9% w/w to 2.0% w/w, 1.85% w/w to 2.05% w/w, or 1.9% w/w to 2.1% w/w. The weight percent of the binder may be from 2.5% w/w to 5.5% w/w, 2.6% w/w to 5.4% w/w, 2.7% w/w to 5.3% w/w, 2.8% w/w to 5.2% w/w, 2.9% w/w to 5.1% w/w, 3.0% w/w to 5.0% w/w, 3.1% w/w to 4.9% w/w, 3.2% w/w to 4.8% w/w, 3.3% w/w to 4.7% w/w, 3.4% w/w to 4.6% w/w, 3.5% w/w to 4.5% w/w, 3.6% w/w to 4.4% w/w, 3.7% w/w to 4.3% w/w, 3.8% w/w to 4.2% w/w, or 3.9% w/w to 4.1% w/w.

In some embodiments, the weight percent of the binder may be 1% w/w to 10% w/w. In some embodiments, the weight percent of the binder may be 1% w/w to 5% w/w. In some embodiments, the weight percent of the binder may be 6% w/w of the total weight of the composition.

In various embodiments, the composition may comprise 10%-60% w/w of the plurality of microcrystalline cellulose (MCC) spheres, 1%-30% w/w octreotide or a pharmaceutically acceptable salt of octreotide, 0.01%-5% w/w magnesium chloride, 1%-20% w/w sodium caprylate, 0.1%-10% w/w polyvinylpyrrolidone, 0.1%-15% w/w EDTA, 0.01%-5% w/w leucine, and 0.01%-10% w/w polysorbate.

In some embodiments, the composition may comprise a plurality of spheres, a peptide or a pharmaceutically acceptable salt of octreotide, magnesium chloride, sodium caprylate, polyvinylpyrrolidone, EDTA, at least one amino acid, and polysorbate. In various embodiments, the composition may comprise 10%-60% w/w of the plurality of microcrystalline cellulose (MCC) spheres, 1%-30% w/w of a peptide or a pharmaceutically acceptable salt, 0.01%-5% w/w magnesium chloride, 1%-20% w/w sodium caprylate, 0.1%-10% w/w polyvinylpyrrolidone, 0.1%-15% w/w EDTA, 0.01%-5% w/w of at least one amino acid, and 0.01%-10% w/w polysorbate. In various embodiments, the composition may comprise 10%-50% w/w of the plurality of microcrystalline cellulose (MCC) spheres, 2%-5% w/w of a peptide or a pharmaceutically acceptable salt, 0.1%-0.5% w/w magnesium chloride, 5%-15% w/w sodium caprylate, 1%-5% w/w polyvinylpyrrolidone, 0.1%-0.7% w/w EDTA, 0.01%-1% w/w of at least one amino acid, and 0.5%-5% w/w polysorbate.

In some embodiments, the composition may comprise a plurality of spheres, octreotide or a pharmaceutically acceptable salt of octreotide, magnesium chloride, sodium caprylate, polyvinylpyrrolidone, EDTA, at least one amino acid, and polysorbate. In some embodiments, the composition may comprise a plurality of microcrystalline cellulose (MCC) spheres, octreotide or a pharmaceutically acceptable salt of octreotide, magnesium chloride, sodium caprylate, polyvinylpyrrolidone, EDTA, leucine, and polysorbate.

In some embodiments, the composition may comprise 10%-60% w/w of the plurality of spheres, 1%-30% w/w octreotide or a pharmaceutically acceptable salt of octreotide, 0.01%-5% w/w magnesium chloride, 1%-20% w/w sodium caprylate, 0.1%-10% w/w polyvinylpyrrolidone, 0.1%-15% w/w EDTA, 0.01%-5% w/w amino acid, and 0.01%-10% w/w polysorbate. In some embodiments, the composition may comprise 10%-60% w/w of the plurality of microcrystalline cellulose (MCC) spheres, 1%-30% w/w octreotide or a pharmaceutically acceptable salt of octreotide, 0.01%-5% w/w magnesium chloride, 1%-20% w/w sodium caprylate, 0.1%-10% w/w polyvinylpyrrolidone, 0.1%-15% w/w EDTA, 0.01%-5% w/w leucine, and 0.01%-10% w/w polysorbate.

In some embodiments, the composition may comprise 10%-50% w/w of the plurality of spheres, 2%-5% w/w octreotide or a pharmaceutically acceptable salt of octreotide, 0.1%-0.5% w/w magnesium chloride, 5%-15% w/w sodium caprylate, 1%-5% w/w polyvinylpyrrolidone, 0.1%-0.7% w/w EDTA, 0.1%-0.5% w/w amino acid, and 0.5%-5% w/w polysorbate. In some embodiments, the composition may comprise 10%-50% w/w of the plurality of microcrystalline cellulose (MCC) spheres, 2%-5% w/w octreotide or a pharmaceutically acceptable salt of octreotide, 0.1%-0.5% w/w magnesium chloride, 5%-15% w/w sodium caprylate, 1%-5% w/w polyvinylpyrrolidone, 0.1%-0.7% w/w EDTA, 0.1%-0.5% w/w leucine, and 0.5%-5% w/w polysorbate.

In some embodiments, the composition may comprise 35%-45% w/w of the plurality of spheres, 2%-4.5% w/w octreotide or a pharmaceutically acceptable salt of octreotide, 0.1%-0.3% w/w magnesium chloride, 7%-12% w/w sodium caprylate, 1%-3% w/w polyvinylpyrrolidone, 0.1%-0.5% w/w EDTA, 0.1%-0.5% w/w amino acid, and 0.5%-1% w/w polysorbate. In some embodiments, the composition may comprise 35%-45% w/w of the plurality of microcrystalline cellulose (MCC) spheres, 2%-4.5% w/w octreotide or a pharmaceutically acceptable salt of octreotide, 0.1%-0.3% w/w magnesium chloride, 7%-12% w/w sodium caprylate, 1%-3% w/w polyvinylpyrrolidone, 0.1%-0.5% w/w EDTA, 0.1%-0.5% w/w leucine, and 0.5%-1% w/w polysorbate.

In some embodiments, the composition may comprise 40% w/w of the plurality of microcrystalline cellulose (MCC) spheres, 3.5% w/w octreotide or a pharmaceutically acceptable salt of octreotide, 0.2% w/w magnesium chloride, 9% w/w sodium caprylate, 2% w/w polyvinylpyrrolidone, 0.4% w/w EDTA, 0.16% w/w leucine, and 0.9% w/w polysorbate. In some embodiments, the composition may comprise 40% w/w of the plurality of microcrystalline cellulose (MCC) spheres, 3.5% w/w octreotide or a pharmaceutically acceptable salt of octreotide, 0.2% w/w magnesium chloride, 9% w/w sodium caprylate, 2% w/w polyvinylpyrrolidone, 0.4% w/w EDTA, 0.16% w/w leucine, and 0.9% w/w polysorbate.

In some embodiments, the composition may comprise about 40% w/w of the plurality of spheres, about 3.5% w/w octreotide or a pharmaceutically acceptable salt of octreotide, about 0.2% w/w magnesium chloride, about 9% w/w sodium caprylate, about 2% w/w polyvinylpyrrolidone, about 0.4% w/w EDTA, about 0.16% w/w amino acid, and about 0.9% w/w polysorbate. In some embodiments, the composition may comprise about 40% w/w of the plurality of microcrystalline cellulose (MCC) spheres, about 3.5% w/w octreotide or a pharmaceutically acceptable salt of octreotide, about 0.2% w/w magnesium chloride, about 9% w/w sodium caprylate, about 2% w/w polyvinylpyrrolidone, about 0.4% w/w EDTA, about 0.16% w/w leucine, and about 0.9% w/w polysorbate.

### Method of Making the Composition

The present disclosure is also directed to a method of making the delayed release composition. The method comprises preparing a plurality of spheres; preparing a first coating solution comprising an amino acid, a divalent salt, a binder, octreotide or a pharmaceutically acceptable salt of octreotide, a permeability enhancer, coating the plurality of spheres with the solution in a fluid bed unit; preparing a second coating solution comprising a stabilizing agent, the binder, and a nonionic surfactant, and divalent salt, coating the plurality of spheres coated with the first coating solution are coated with the second coating solution; encapsulating the coated plurality of spheres into a capsule; preparing an enteric coating solution; and coating the capsule with the enteric coating solution. Also provided herein are methods of reducing impurities in an octreotide composition. The method may include adding an amino acid to a delayed release composition of octreotide.

In some embodiments, the amino acid may comprise leucine, cysteine, tryptophan, methionine, lysine, arginine, tyrosine, glycine, histidine or any combination thereof.

In some embodiments, the spheres may comprise microcrystalline cellulose (MCC) spheres, sugar spheres, or any other suitable spheres. In further embodiments, the plurality of particles may comprise a plurality of any suitable delayed release beads.

In some embodiments, the divalent salts may comprise salts of calcium, magnesium, zinc, or iron. Examples of calcium salts include but are not limited to calcium carbonate, calcium citrate, or calcium chloride. Examples of magnesium salts include but are not limited to magnesium sulfate, magnesium stearate, or magnesium chloride. Examples of divalent zinc salts include but are not limited to zinc sulfate, zinc acetate, or zinc chloride.

In some embodiments, the binder may comprise a natural binder, a synthetic binder, a synthetic polymer, or other materials. Natural binders include but are not limited to starch and its derivatives, cellulose (e.g. microcrystalline cellulose), gelatin, or acacia and guar gum. Synthetic binders include but are not limited to polyvinylpyrrolidone (PVP), hydroxypropyl methylcellulose (HPMC), or polyethylene glycol (PEG). Synthetic polymers include but are not limited to ethylcellulose or polyvinyl alcohol (PVA). Other binders include magnesium stearate or lactose. In some embodiments, the binder may comprise polyvinylpyrrolidone (PVP). In various embodiments, the binder may comprise more than one form of PVP. In additional embodiments the PVP may comprise PVP-12, PVP-17 (Povidone K17), PVP K-90, and PVP K-30. In some embodiments, the binder may comprise PVP-12, PVP-17, or both.

In some embodiments, the permeability enhancer may be a fatty acid or fatty acid derivative, a surfactant, a stabilizing agent, a chemical modifier, or an enzyme inhibitor. Examples of the fatty acid or fatty acid derivative include but are not limited to sodium caprylate, oleic acid, or lauric acid. Examples of the surfactants include but are not limited to sodium lauryl sulfate (SLS), polysorbates, or Triton X-100. Examples of chemical modifiers include but are not limited to cyclodextrins, azone, or menthol. Examples of enzyme inhibitors include but are not limited to protease inhibitors or P-Glycoprotein inhibitors. In some embodiments, the permeability enhancer may comprise sodium caprylate. In some embodiments, the permeability enhancer may comprise sodium caprylate, polysorbate 80, EDTA, or any combination thereof.

In some embodiments, the stabilizing agent may comprise a stabilizing agent for example, ethylenediaminetetraacetic acid (EDTA), deferoxamine, dimercaprol (British Anti-Lewisite), penicillamine, succimer (dimercaptosuccinic Acid, DMSA), thiamine monophosphate (thiamine chelate), trientine, calcium disodium EDTA, ferric citrate, or any other suitable stabilizing agent.

The preparing of the first coating solution may include adding and mixing an amino acid, isopropanol, magnesium chloride, polyvinylpyrrolidone, octreotide acetate, and/or sodium caprylate. In some examples, the amino acid may be leucine.

The preparing the second solution may include adding EDTA, heating, and cooling the second solution before adding isopropanol. The preparing the second solution may also comprise adding polyvinylpyrrolidone, polysorbate, sodium caprylate, and mixing.

The preparing a plurality of spheres step may include charging in a fluid bed system, using a spray process, and screening the resulting spheres. In some embodiments, the spray process utilizes a nozzle size of about 0.2 mm to about a 0.1 mm, pump speed of about 1-10 rpm, a spray rate of 0.2-5 g/min, an inlet air temperature of 40 °C to 60 °C, and product temperature of 35 °C-50 °C.

In some embodiments, the first solution may be a coating solution comprising octreotide acetate, magnesium chloride, PVP-12, and sodium caprylate in isopropyl alcohol (IPA) and water. The isopropyl alcohol and water may be in a ratio of about 1:1. The second solution may be a second coating solution comprising sodium EDTA, PVP-12, sodium caprylate, and polysorbate 80 in isopropyl alcohol and water. The isopropyl alcohol and water may be in a ratio of about 1:1. The method may also comprise applying an enteric coating to the capsule therefore forming the delayed release capsule composition.

In some embodiments, the method comprises applying an enteric coating to the capsule. In some embodiments of the method, the enteric coating solution comprises enteric coating polymer (aqueous acrylic enteric systems sold under the trademark Acryl-EZE®) and triethyl citrate (TEC) in deionized water (DI).

In multiple embodiments of the method, including an addition of an amino acid (e.g., leucine) reduced peptide impurities in the finished composition compared to methods that did not include addition of an amino acid.

Intestinal absorption of peptides (>3 amino acids) may be limited by low intestinal permeability and intestinal enzymatic degradation. Peptide drugs may be transported across intestinal cell epithelium by paracellular and transcellular transport. Paracellular transport is the transfer of substances across an epithelium (e.g., the intestinal epithelium) by passing through the intercellular space between cells. Paracellular transport may be limited by the ability of a substance to pass through tight junctions. Calcium concentrations may regulate the transport function of tight junctions. In some embodiments, the disclosed compositions may include a stabilizing agent wherein the composition may cause chelation of minerals including calcium, potentially allowing tight junctions to open thereby increasing paracellular transport.

Transcellular transport is the transfer of substances through a cell either through active transport, passive transport, or transcytosis.

In some embodiments, the disclosed compositions may be transported across the intestinal epithelium by a mix of paracellular and transcellular transport. In some embodiments, an equivalent amount of the composition may be transported via paracellular transport as compared to transcellular transport. In some embodiments, the composition may be transported by increased paracellular transport as compared to transcellular transport. In an alternative embodiment, the composition may be transported by an increased transcellular transport as compared to paracellular transport.

### EXAMPLES

The following examples are offered to illustrate, but not to limit the disclosure.

### Example 1: Octreotide Composition 1

A 20 mg octreotide acetate composition was prepared. Table 2 shows an octreotide acetate composition (composition 1).

TABLE 2

| | Composition 1 | |
|---|---|---|
| Components | mg | % w/w |
| Coated MCC Spheres | 390 | 64.27 |
| Octreotide acetate | 22.7 | 3.74 |
| Capsule | 105 | 17.30 |
| Methacrylic Acid Co[[P]]polymer | 35.64 | 5.87 |
| Talc | 33.19 | 5.47 |
| Titanium Dioxide | 13.37 | 2.20 |
| Triethyl Citrate | 4.28 | 0.71 |
| Colloidal Silicon Dioxide | 1.11 | 0.18 |
| Sodium Bicarbonate | 1.07 | 0.18 |
| Sodium Lauryl Sulfate | 0.45 | 0.07 |
| Total | 606.81 | 100 |

### Example 2: Octreotide Composition 2

A 20 mg octreotide acetate composition was prepared. Table 3 shows an octreotide acetate composition (composition 2) that may be formulated into a delayed release capsule.

| | Composition 2 | |
|---|---|---|
| Components | mg | % w/w |
| Coated MCC Spheres | 390 | 61.75 |
| Octreotide acetate | 22.7 | 3.59 |
| Capsule | 105 | 16.63 |
| Methacrylic Acid Copolymer | 45.54 | 7.21 |
| Talc | 42.41 | 6.72 |
| Titanium Dioxide | 17.08 | 2.70 |
| Triethyl Citrate | 5.46 | 0.86 |
| Colloidal Silicon Dioxide | 1.42 | 0.22 |
| Sodium Bicarbonate | 1.37 | 0.22 |
| Sodium Lauryl Sulfate | 0.57 | 0.09 |
| Total | 631.55 | 100.00 |

### Example 3: Octreotide Composition 3

A 20 mg octreotide acetate composition was prepared. Table 4 shows an octreotide acetate composition (composition 3) that may be formulated into a delayed release capsule.

TABLE 4

| | Composition 3 | |
|---|---|---|
| Components | mg | % w/w |
| Coated MCC Spheres | 390 | 64.27 |
| Octreotide acetate | 22.7 | 3.74 |
| Capsule | 105 | 17.30 |
| Methacrylic Acid | 35.64 | 5.87 |
| Copolymer | | |
| Talc | 33.19 | 5.47 |
| Titanium Dioxide | 13.36 | 2.20 |
| Triethyl Citrate | 4.28 | 0.71 |
| Colloidal Silicon Dioxide | 1.11 | 0.18 |
| Sodium Bicarbonate | 1.07 | 0.18 |
| Sodium Lauryl Sulfate | 0.45 | 0.07 |
| Total | 606.8 | 100.00 |

### Example 4: Octreotide Composition 4

A 20 mg octreotide acetate delayed release composition was prepared. Table 5 shows an octreotide acetate delayed release composition (composition 4).

TABLE 5

| | Composition 4 | |
|---|---|---|
| Components | mg | % w/w |
| MCC Spheres | 258.08 | 40.11 |
| Octreotide Acetate | 22.70 | 3.53 |
| Magnesium Chloride | 1.26 | 0.20 |
| EDTA | 2.50 | 0.39 |
| Polysorbate 80 | 5.66 | 0.88 |
| PVP-12 | 12.60 | 1.96 |
| Povidone K17 | 27.19 | 4.23 |
| Sodium Caprylate | 60.01 | 9.33 |
| Capsule | 105.00 | 16.32 |
| Acryl-Eze ® 93A Clear | 134.98 | 20.98 |
| Triethyl Citrate | 13.51 | 2.10 |
| Total | 643.48 | 100.00 |

### Example 5: Octreotide Composition Comprising Leucine

A 20 mg octreotide acetate delayed release composition was prepared. Table 6 shows an octreotide acetate delayed release composition with leucine (composition 5).

TABLE 6
Components in an octreotide acetate delayed release
composition comprising leucine (composition 5).

| | Composition 5 | |
|---|---|---|
| Components | mg | % w/w |
| MCC Spheres | 258.08 | 40.09 |
| Octreotide Acetate | 22.70 | 3.53 |
| Magnesium Chloride | 1.26 | 0.20 |
| EDTA | 2.50 | 0.39 |
| Polysorbate 80 | 5.66 | 0.88 |
| Leucine | 1.00 | 0.16 |
| Povidone K12 | 12.60 | 1.96 |
| Povidone K17 | 26.41 | 4.10 |
| Sodium Caprylate | 60.01 | 9.32 |
| Capsule | 105.00 | 16.31 |
| Acryl-Eze ® 93A Clear | 134.98 | 20.97 |
| Triethyl citrate | 13.51 | 2.10 |
| Total | 643.71 | 100.00 |

### Example 6: Octreotide Composition Comprising Cysteine

A 20 mg octreotide acetate composition was prepared. Table 7 shows an octreotide acetate composition comprising cysteine (composition 6) that may be formulated into a delayed release capsule.

TABLE 7

| | Composition 6 | |
|---|---|---|
| Components | mg | % w/w |
| MCC Spheres | 258.08 | 66.17 |
| Octreotide Acetate | 22.70 | 5.82 |
| Magnesium Chloride | 1.26 | 0.32 |
| EDTA | 2.50 | 0.64 |
| Polysorbate 80 | 5.66 | 1.45 |
| Cysteine | 6.00 | 1.54 |
| PVP-12 | 12.60 | 3.23 |
| Povidone K17 | 21.21 | 5.44 |
| Sodium Caprylate | 60.01 | 15.39 |
| Total | 390.00 | 100.00 |

### Example 7: Composition of First Coating Layer of Composition 5 (Example 5)

Table 8 shows the composition of a first coating layer in composition 5.

TABLE 8

| | Composition 5 | | |
|---|---|---|---|
| | | % w/w in | Batch |
| Coating Layer 1-Active Layer | | coating | Qty |
| Raw Materials | % w/w | solution | (g) |
| MCC Spheres (VIVAPUR ® 500) | 83.25 | | 155.00 |
| Octreotide Acetate | 7.32 | 43.53 | 13.63 |
| Magnesium Chloride | 0.41 | 2.41 | 0.76 |
| Leucine | 0.32 | 1.92 | 0.60 |
| Povidone K12 | 1.80 | 10.70 | 3.35 |
| Povidone K17 | 3.74 | 22.23 | 6.96 |
| Sodium Caprylate | 3.23 | 19.20 | 6.01 |
| Isopropyl Alcohol | | | 140.34 |
| Water | | | 140.34 |
| Total | 100.07 | 100.00 | 312.00 |

### Example 8: Composition of First Coating Layer of Composition 4 (Example 4)

Table 9 shows the composition of a first coating layer in composition 4.

TABLE 9

| | Composition 4 | | |
|---|---|---|---|
| Coating Layer 1-Active | | % w/w in | Batch |
| Layer | | coating | Qty |
| Raw Materials | % w/w | solution | (g) |
| MCC Spheres | 83.25 | | 155.00 |
| (VIVAPUR ® 500) | | | |
| Octreotide Acetate | 7.32 | 43.72 | 13.63 |
| Magnesium Chloride | 0.41 | 2.42 | 0.76 |
| Leucine | — | — | — |
| Povidone K12 | 1.80 | 10.75 | 3.35 |
| Povidone K17 | 3.99 | 23.82 | 7.43 |
| Sodium Caprylate | 3.23 | 19.28 | 6.01 |
| Isopropyl Alcohol | | | 140.34 |
| Water | | | 140.34 |
| Total | 100.00 | 100.00 | 311.86 |

### Example 9: Composition of Second Coating Layer of Composition 5 (Example 5)

Table 10 shows the composition of a second coating layer in composition 5.

TABLE 10

| | Composition 5 | | |
|---|---|---|---|
| | | % w/w of | Batch |
| Coating Layer 2-PE Layer | | coating | Qty |
| Raw Materials | % w/w | solution | (g) |
| Drug Layer Coated Beads | 79.49 | | 150.00 |
| EDTA (disodium salt) | 0.64 | 3.12 | 1.21 |
| Povidone K12 | 1.80 | 8.78 | 3.40 |
| Povidone K17 | 3.80 | 18.53 | 7.17 |
| Polysorbate 80 | 1.45 | 7.07 | 2.74 |
| Sodium Caprylate | 12.82 | 62.51 | 24.19 |
| Isopropyl alcohol | | | 174.16 |
| Water | | | 174.16 |
| Total | 100.00 | 100.00 | 387.03 |

### Example 10: Composition of Second Coating Layer of Composition 4 (Example 4)

Table 11 shows the composition of a second coating layer in composition 4.

TABLE 11

| | Composition 4 | | |
|---|---|---|---|
| | | % w/w of | Batch |
| Coating Layer 2-PE Layer | | coating | Qty |
| Raw Materials | % w/w | solution | (g) |
| Drug Layer Coated Beads | 79.49 | | 150.00 |
| EDTA (disodium salt) | 0.64 | 3.12 | 1.21 |
| Povidone K12 | 1.80 | 8.78 | 3.40 |
| Povidone K17 | 3.80 | 18.53 | 7.17 |
| Polysorbate 80 | 1.45 | 7.07 | 2.74 |
| Sodium Caprylate | 12.82 | 62.51 | 24.19 |
| IPA | | | 174.16 |
| Water | | | 174.16 |

Numerous examples are provided herein to enhance the understanding of the present disclosure. A specific set of statements are provided as follows.

Statement 1: A delayed release composition comprising a plurality of spheres; an octreotide or a pharmaceutically acceptable salt of octreotide; a permeability enhancer; a stabilizing agent; a divalent salt; and a binder.

Statement 2: The delayed release composition of statement 1, wherein the spheres are microcrystalline cellulose (MCC).

Statement 3: The delayed release composition of statement 1, wherein the permeability enhancer comprises sodium caprylate, EDTA, polysorbate 80, or any combination of thereof.

Statement 4: The delayed release composition of statement 1, wherein the stabilizing agent comprises one or more amino acids.

Statement 5: The delayed release composition of statement 4, wherein the amino acid comprises leucine, cysteine, tryptophan, methionine, lysine, arginine, tyrosine, glycine, histidine, or any combination thereof.

Statement 6: The delayed release composition of statement 1, wherein the divalent salt comprises magnesium chloride.

Statement 7: The delayed release composition of statement 1, wherein the binder comprises polyvinylpyrrolidone.

Statement 8: The delayed release composition of statement 1, wherein the composition is formulated as a capsule and the capsule is coated with an enteric coating.

Statement 9: The delayed release composition of statement 4, wherein the ratio of octreotide or salt thereof to amino acid comprises 1:1 to 50:1, 2:1 to 50:1, 3:1 to 50:1, or 4:1 to 50:1 on a w/w basis.

Statement 10: The delayed release composition of statement 5, wherein the ratio of octreotide or salt thereof to leucine is about 1:22.

Statement 11: The delayed release composition of statement 1 comprising about 10% about 60% w/w of the plurality of microcrystalline cellulose (MCC) spheres; about 1% about 30% w/w octreotide or a pharmaceutically acceptable salt of octreotide; about 0.1% about 1% w/w magnesium chloride; about 1% about 20% w/w sodium caprylate; about 0.1% about 10% w/w polyvinylpyrrolidone; about 0.1% about 15% w/w EDTA; about 0.01% about 5% w/w leucine; and about 0.01% about 10% w/w polysorbate.

Statement 12: The delayed release composition of statement 11 comprising 20%-50% w/w of the plurality of microcrystalline cellulose (MCC) spheres; 1%-10% w/w octreotide or a pharmaceutically acceptable salt of octreotide; 0.1%-0.5% w/w magnesium chloride; 5%-15% w/w sodium caprylate; 1%-5% w/w polyvinylpyrrolidone; 0.1%-1% w/w EDTA; 0.01%-1% w/w leucine; and 0.5%-5% w/w polysorbate.

Statement 13: A method of making a delayed release composition, the method comprising preparing a plurality of spheres; preparing a first coating solution comprising an amino acid, a divalent salt, a binder, octreotide or a pharmaceutically acceptable salt of octreotide, and a permeability enhancer; coating the plurality of spheres with the first coating solution in a fluid bed unit; preparing a second coating solution comprising a stabilizing agent, the binder, and a nonionic surfactant, and divalent salt; coating the plurality of spheres coated with the first coating solution with the second coating solution; encapsulating the coated plurality of spheres into a capsule; preparing an enteric coating solution; and coating the capsule with the enteric coating solution.

Statement 14: The method of statement 13, wherein the preparing of the first coating solution comprises adding and mixing an amino acid, isopropanol, magnesium chloride, polyvinylpyrrolidone, octreotide acetate, and/or sodium caprylate.

Statement 15: The method of statement 13, wherein the preparing the second solution comprises adding EDTA, heating, and cooling the second solution before adding isopropanol.

Statement 16: The method of statement 14, wherein the preparing the second solution further comprises adding polyvinylpyrrolidone, polysorbate, sodium caprylate, and mixing.

Statement 17: The method of statement 13, wherein the enteric coating solution comprises enteric coating polymer (Acryl-EZE®) and triethyl citrate (TEC) in deionized water (DI).

Statement 18: The method of statement 13, wherein the first coating solution comprises a coating solution comprising octreotide acetate, magnesium chloride, PVP-12, and sodium caprylate in isopropyl alcohol and water.

Statement 19: A composition comprising a peptide or a pharmaceutically acceptable salt of a peptide; and an amino acid comprising leucine, cysteine, aspartate, phenylalanine, glutamine, or a combination thereof, wherein the ratio of the peptide or salt thereof to amino acid is 1:1 to 50:1, 2:1 to 50:1, 3:1 to 50:1, or 4:1 to 50:1 on a w/w basis.

Statement 20: The composition of statement 19, wherein the composition is formulated as a delayed release capsule.

Statement 21: The composition of statement 20, wherein the ratio of the peptide or salt thereof to amino acid is about 1:22.

Statement 22: The composition of statement 21, wherein the amino acid is leucine.

Statement 23: The composition of statement 21, wherein the peptide comprises octreotide, leuprolide, oxytocin, goserelin, triptorelin, buserelin, deslorelin, vasopressin, lypressin, somatostatin, melanocyte-stimulating hormone, angiotensin II, or terlipressin.

1. A delayed release dispersion composition which dispersion is not an oil, and which composition comprising:
a plurality of spheres consisting of microcrystalline cellulose (MCC);
an octreotide or a pharmaceutically acceptable salt of octreotide;
a permeability enhancer selected from the group consisting of sodium caprylate, polyoxyethylene (20) sorbitan monooleate, or both;
ethylenediaminetetraacetic acid (EDTA);
a divalent salt; and
a binder consisting of polyvinylpyrrolidone (PVP);
wherein the plurality of spheres comprises a first coating layer and a second coating layer;
wherein the first coating layer comprises the divalent salt, the binder, the octreotide or a pharmaceutically acceptable salt of octreotide, and the permeability enhancer;
wherein the second coating layer comprises the EDTA, the binder, and the divalent salt; and
wherein EDTA is present only in the second coating layer.

2. (canceled)

3. (canceled)

4. The delayed release composition of claim 1, wherein the first coating layer further comprises one or more amino acids; and wherein a ratio of octreotide or salt thereof to the one or more amino acids is in the range of from 1:1 to 50:1 on a w/w basis.

5. The delayed release composition of claim 4, wherein the amino acid is selected from the group consisting of leucine, cysteine, tryptophan, methionine, lysine, arginine, tyrosine, glycine, histidine, and any combination thereof.

6. The delayed release composition of claim 1, wherein the divalent salt is magnesium chloride.

7. (canceled)

8. The delayed release composition of claim 1, wherein the plurality of spheres are encapsulated in a capsule and the capsule is coated with an enteric coating.

9. The delayed release composition of claim 4, wherein the ratio of octreotide or salt thereof to the one or more amino acids is in the range of from 2:1 to 50:1 on a w/w basis.

10. (canceled)

11. (canceled)

12. (canceled)

13. A method of making a delayed release composition of claim 1, the method comprising:
preparing a plurality of spheres;
preparing a first coating solution comprising an amino acid, a divalent salt, a binder, octreotide or a pharmaceutically acceptable salt of octreotide, and a permeability enhancer;
coating the plurality of spheres with the first coating solution in a fluid bed unit;
preparing a second coating solution comprising a stabilizing agent, the binder, and a nonionic surfactant, and divalent salt;
coating the plurality of spheres coated with the first coating solution with the second coating solution;
encapsulating the coated plurality of spheres into a capsule;
preparing an enteric coating solution; and
coating the capsule with the enteric coating solution.

14. The method of claim 13, wherein the preparing of the first coating solution comprises adding and mixing an amino acid, isopropanol, magnesium chloride, PVP, octreotide acetate, and/or sodium caprylate.

15. The method of claim 13, wherein the preparing the second solution comprises adding ethylenediaminetetraacetic acid (EDTA), heating, and cooling the second solution before adding isopropanol.

16. The method of claim 14, wherein the preparing the second solution further comprises adding PVP, polysorbate, sodium caprylate, and mixing.

17. The method of claim 13, wherein the enteric coating solution comprises an aqueous methacrylic acid copolymer dispersion and triethyl citrate (TEC) in deionized water (DI).

18. The method of claim 13, wherein the first coating solution comprises a coating solution comprising octreotide acetate, magnesium chloride, PVP-12, and sodium caprylate in isopropyl alcohol and water.

19. A composition comprising:
a peptide or a pharmaceutically acceptable salt of a peptide; and
an amino acid comprising leucine, cysteine, aspartate, phenylalanine, glutamine, or a combination thereof,
wherein ratio of the peptide or salt thereof to amino acid is from 1:1 to 50:1, 2:1 to 50:1, 3:1 to 50:1, or 4:1 to 50:1 on a w/w basis.

20. The composition of claim 19, wherein the composition is formulated as a delayed release capsule.

21. The composition of claim 20, wherein the ratio of the peptide or salt thereof to amino acid is about 1:22.

22. The composition of claim 21, wherein the amino acid is leucine.

23. The composition of claim 21, wherein the peptide comprises octreotide, leuprolide, oxytocin, goserelin, triptorelin, buserelin, deslorelin, vasopressin, lypressin, somatostatin, melanocyte-stimulating hormone, angiotensin II, or terlipressin.

24. (canceled)

25. The delayed release composition of claim 5, wherein the amino acid is leucine.

26. The delayed release composition of claim 25, wherein ratio of octreotide or salt thereof to leucine is about 1:22


SEQUENCE LISTING (13 sequences)

SEQ ID NO: 1 - protein, 8 aa, synthetic construct
FCFWKTCT

SEQ ID NO: 2 - protein, 9 aa, synthetic construct
XHWSYLLRP

SEQ ID NO: 3 - protein, 9 aa, synthetic construct
CYIQNCPLG

SEQ ID NO: 4 - protein, 9 aa, synthetic construct
XHWSYXLRP

SEQ ID NO: 5 - protein, 10 aa, synthetic construct
XHWSYWLRPG

SEQ ID NO: 6 - protein, 9 aa, synthetic construct
XHWSYXLRP

SEQ ID NO: 7 - protein, 9 aa, synthetic construct
XHWSYWLRP

SEQ ID NO: 8 - protein, 9 aa, synthetic construct
CYFQNCPKG

SEQ ID NO: 9 - protein, 9 aa, synthetic construct
CYFQNCPKG

SEQ ID NO: 10 - protein, 14 aa, synthetic construct
AGCKNFFWKTFTSC

SEQ ID NO: 11 - protein, 13 aa, synthetic construct
SYSMEHFRWGKPV

SEQ ID NO: 12 - protein, 8 aa, synthetic construct
DRVYIHPF

SEQ ID NO: 13 - protein, 12 aa, synthetic construct
GGGCYFQNCPKG